(19) United States
(12) Patent Application Publication
(10) Pub. No.: US 2012/0127823 A1
(43) Pub. Date: May 24, 2012

(54) HIGH-PRESSURE HOMOGENIZATION WITH A SILICON NITRIDE VALVE

(75) Inventor: Mostafa Nakach, Paris (FR)

(73) Assignee: SANOFI, Paris (FR)

(21) Appl. No.: 13/320,191

(22) PCT Filed: May 10, 2010
(86) PCT No.: PCT/FR2010/050902

§ 371 Date: Feb. 6, 2012

(30) Foreign Application Priority Data
May 13, 2009 (FR) 0902290

(51) Int. Cl. B01F 5/06; B82Y 5/00

(52) U.S. Cl. 366/176.3; 977/786

(57) ABSTRACT

The invention relates to the use of a homogenisation valve that comprises a flap gate (1), an impact ring (3) and a seat in order to prepare, using high-pressure valve technology, a nanosuspension of a solid pharmaceutically active principle, characterized in that the material constituting the flap gate, the seat and optionally the impact ring and/or the outer surface of at least one of said elements includes sintered or hot-pressed silicon nitride as the main component. The invention also relates to a method for preparing a nanosuspension of a solid pharmaceutically active principle using the high-pressure valve homogenization technology.

The present application relates to the use of a homogenizing valve used in piston-gap high-pressure technology for preparing a nanosuspension of a solid pharmaceutical active principle. The invention also relates to a method using said valve.

### TECHNICAL FIELD

High-pressure homogenization (HPH) technology is used in galenics to obtain nanosuspensions of particles of a solid pharmaceutical active principle that exhibits very low solubility in water. The particles are characterized by a mean diameter d₅₀<500 nm and are stabilized by at least one stabilizer/surfactant, So-called “piston-gap” high-pressure technology (which uses a valve) which forms the subject of the present invention was developed by R. H. Müller and is described in U.S. Pat. No. 5,858,410, EP 1964605 and in the articles “Dissocubes®—a novel formulation for poorly soluble and poorly available drugs” Müller, p. 135 from the book “Modified-release drug delivery technology”, 2002, isbn 0-8247-0869-5 or J. Pharm. Pharmaco. 2004, 56, 827-840. This technology is also described in Chapter 9.2 of the book “Emulsions and nanosuspensions for the formulation of poorly soluble drugs” Medpharm, 1998, isbn 3-88763-069-6.

### TECHNICAL PROBLEM

The problem addressed is that of being able to have a piston-gap high-pressure homogenization technology that can be used to prepare nanosuspensions without any contamination from grinding residue and which uses robust tooling capable of operating at a high flow rate and which demands the lowest possible amount of maintenance. The Applicant company has discovered that this problem can be solved by using sintered or hot-pressed silicon nitride as the material from which to make the valve piston, the valve seat and possibly the impact ring or the exterior surface of said elements.

### PRIOR ART

JP 1028282 describes sintered ceramics (Si₃N₄, SiC, Si₅AlON₇, etc.) that have good mechanical and erosion-resistance properties.

WO 2007/148237 describes a valve for a piston-gap type homogenizer.

WO 2005/097308 describes a piston-gap homogenizer of which one of the elements (“plunger 5”), which is not a valve, is made of silicon nitride Si₃N₄.

EP 1964605 describes a piston-gap homogenizer of which one of the elements (12c) is made of carbide (WC—Co, WC—TiC—Co, WC—TiC—Ta(Nb)C—Co, etc.).

### BRIEF DESCRIPTION OF THE INVENTION

The invention relates to the use of a homogenizing valve consisting of a valve piston, of an impact ring and of a valve seat for the preparation of a nanosuspension of a solid pharmaceutical active principle using piston-gap high-pressure technology, characterized in that the material from which (i) the valve seat, partially, completely or the exterior surface thereof, (ii) the valve piston, completely or the exterior surface thereof (iii) and possibly the impact ring, completely or the exterior surface thereof, are made comprises, by way of predominant component, sintered or hot-pressed silicon nitride.

The invention also relates to a method of preparing a nanosuspension of a solid pharmaceutical active principle using piston-gap high-pressure homogenization technology, involving:
pumping a dispersion of the active principle in a liquid phase to which at least one stabilizer/surfactant has been added;
compressing said dispersion to a pressure ranging from 100 to 2000 bar;
expanding said dispersion through a homogenization valve consisting of a valve piston, of an impact ring and of a valve seat, characterized in that the material from which (i) the valve seat, partially, completely or the exterior surface thereof, (ii) the valve piston, completely or the exterior surface thereof (iii) and possibly the impact ring, completely or the exterior surface thereof comprises, by way of predominant component, sintered or hot-pressed silicon nitride.

### FIGURES

FIG. 1: a diagram of a high-pressure homogenizing valve and the dispersion flow (inlet, outlet).

FIG. 2: a drawing of the silicon nitride homogenizing valve according to one of the embodiments of the invention used in the examples. The indicated dimensions are given in millimeters and illustrate one embodiment of the valve according to the invention.

### DETAILED DESCRIPTION

### Definitions

nanosuspension: suspension of nanoparticles;
nanoparticles: particles of a solid compound with a mean diameter d50 (determined by laser scattering) of <1000 nm;

Because this is piston-gap high-pressure technology, it can be used to prepare a nanosuspension from a dispersion of a solid pharmaceutical active principle in a liquid phase, the initial mean diameter d50 of which is higher than the mean diameter d50 of the nanosuspension. The liquid phase generally consists of pure water although pharmaceutically acceptable solvents such as ethanol for example may also be added. The initial mean diameter d50 is preferably <25 μm in order to avoid blocking the gap between the valve seat and the valve piston. The stability of the nanosuspension is ensured using at least one stabilizer/surfactant which will be chosen according to the pharmaceutical active principle and according to the particle size of the nanosuspension.

Piston-gap high-pressure technology involves using a piston pump to impose a high pressure (of the order of 100 to 2000 bar) on the dispersion, then expanding the dispersion through a homogenizing valve (described later on). The principle of reducing the size is based firstly on the density of energy generated by the inter-particle impacts and by collision between the particles and the valve piston and with the impact ring and, secondly, on the energy generated by cavitation and by turbulence. Cavitation is caused by the rapid expansion of the liquid which causes microbubbles of vapor to form. Devices that employ this technology are marketed for example by the company APV Gaulin GmbH.

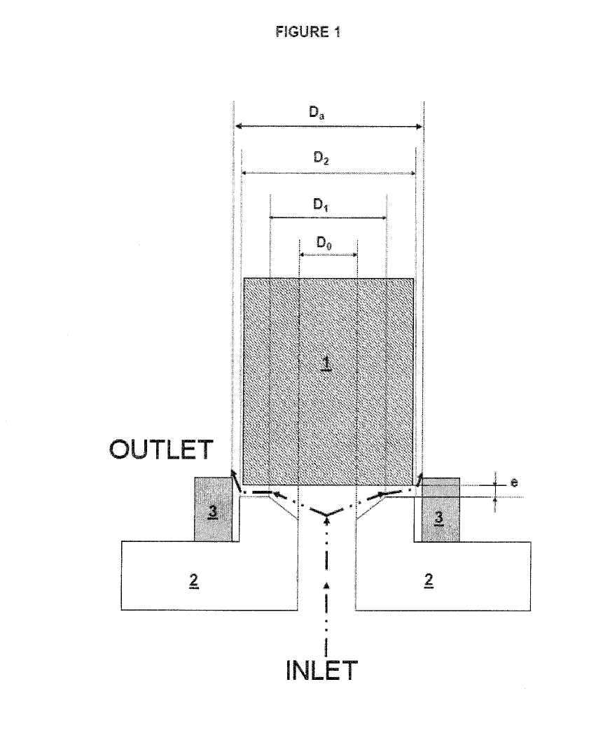

The homogenizing valve consists of 3 elements: a valve piston (1), a valve seat (2) and an impact ring (3) (see FIG. 1). It has been found that the technical problems described hereinabove can be solved if (i) the valve seat (2) is made partly, completely or the exterior surface thereof, from the silicon-nitride-based material described hereinafter and if, (ii) the valve piston (1) is made completely or the exterior surface thereof, from said material. This silicon-nitride-based material is strong enough to allow the preparation of the nanosuspension for a long period of time and without the need to dismantle the homogenizing valve in order to change one of the elements thereof. The impact ring (3) or the exterior surface thereof may also be made of a similar material.

According to one of the alternative forms of the invention, it is possible for just the external surface of the valve piston (1) and/or of the valve seat (2), and/or possibly of the impact ring (3) to be made of said silicon-nitride-based material, the core of said elements for its part being made of some other material that does not have the same impact-resistant and abrasion-resistant mechanical properties. For example:
E1—the exterior surface of the valve piston and the exterior surface of the valve seat are made of said silicon-nitride-based material;
E2—the valve piston is made completely from said silicon-nitride-based material and the exterior surface of the valve seat is made from said silicon-nitride-based material;
E3—the exterior surface of the valve piston is made from said silicon-nitride-based material and the valve seat is made completely from said silicon-nitride-based material.

For these three embodiments E1. E2, E3 above, the ring or the exterior surface thereof may be made of said silicon-nitride-based material or from some other material (as illustrated in the examples: tungsten carbide, etc.).

As far as the valve seat is concerned, this may actually be made completely from the silicon-nitride-based material described below. It is also possible for just one of the parts of the valve seat to be made of said material. Notably and for example, the internal part (6c, 6d) of the valve seat is made of said silicon-nitride-based material, and the external part (6a, 6b) of the valve seat is made of some other material that does not have the same impact-resistant and abrasion-resistant mechanical properties. This other material may notably be based on stainless steel (on steels made of an alloy of iron, chromium, nickel and other ores that afford it a certain degree of corrosion resistance). For this embodiment, the surface of the valve piston is made of a silicon-nitride-based material or preferably the valve piston is made completely of the silicon-nitride-based material. For this same embodiment, the ring may be made of said silicon-nitride-based material or of some other material (as illustrated in the examples: tungsten carbide, etc.).

In practice the starting point is to prepare, in the liquid phase, a dispersion of the solid pharmaceutical active principle, the initial mean diameter d50 of which is preferably <25 μm. At least one stabilizer/surfactant is added to this dispersion. The dispersion is then pumped to the high-pressure homogenizer and compressed to a pressure ranging from 100 to 2000 bar, and is then expanded through the homogenizing valve described above. The compression is performed by a piston pump. A recirculation loop allows the dispersion to be recirculated through the homogenizing valve several times, if necessary.

In terms of the silicon-nitride-based material, this by way of predominant component contains sintered silicon nitride (or SSN) or hot-pressed silicon nitride (or HPSN which stands for high-pressure silicon nitride). For preference, the material contains over 75% (by weight), advantageously over 80%, preferably over 85% sintered or hot-pressed silicon nitride. It may contain other components the function of which is to enhance the mechanical properties of the silicon nitride or to be sintering agents, for example Al₂O₃, Y₂O₃, TiO₂, Nd₂O₃. The material preferably contains, by weight, from 80 to 90% sintered or hot-pressed silicon nitride and from 0 to 20% of component(s) chosen from Al₂O₃, Y₂O₃, TiO₂ or Nd₂O₃.

One example of a silicon-nitride-based material that can be used is KERSIT® 301 which is a hot-pressed silicon nitride developed by C.T.Desrnarquest (from the Saint Gobain group) and of which the composition by weight and properties are as follows: Si₃N₄: 88.5%; Al₂O₃, Y₂O₃, Nd₂O₃, TiO₂: 11.5%; density>3.25; bending strength>800 MPa; hardness: 1450 Hv; toughness: 7 MPa·m^{1/2}),

### EXAMPLES

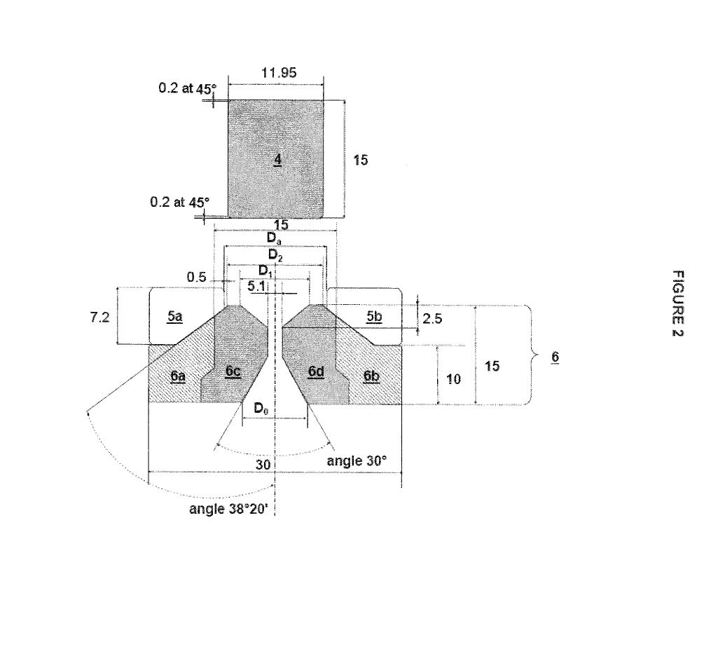

Three valves made of 3 different materials were tested:
one valve made of zirconium oxide (supplied by Niro-Soavi);
one valve made of tungsten carbide coated with titanium nitride (supplied by Niro-Soavi);
one valve made of silicon nitride manufactured in the KERSIT® 301 grade described above, and depicted in FIG. 2.

Use was made of two Niro-Soavi homogenizers: NS2006 (35 l/h, 1500 bar) and NS3024 (300 l/h, 1500 bar). Each of the valves is made up of a valve seat (2), a valve piston (or impact head) (1) and an impact ring (3) (see FIG. 1). The dimensions and relative configuration of each of the valves are described in Table I.

Regarding the valve of FIG. 2, the valve piston (4) is made of silicon nitride. The stationary impact ring (5) is made of tungsten carbide (5a,5b), the valve seat (6) is made of stainless steel (6a,6b) and of silicon nitride (6c,6d).

TABLE I

| | | valve made of | |
|---|---|---|---|
| | valve made | tungsten carbide | |
| | of zirco- | and coated with | valve made of |
| | nium oxide | titanium nitride | silicon nitride |
| geometric shape of | straight tube | straight tube | convergent- |
| valve seat | followed by | followed by | divergent as |
| | divergent | divergent | per FIG. 2 |
| inlet diameter D₀ | 7.98 | 5 | 9 |
| [mm] | | | |
| inside diameter of | 11 | 10.9 | 11 |
| divergent D₁ [mm] | | | |
| outside diameter of | 12 | 12.1 | 12 |
| divergent D₂ [mm] | | | |
| inside diameter of | 12.48 | 12.4 | 12.35 |
| impact ring Dₐ [mm] | | | |

Tests were first of all carried out at a pressure of 1400 bar and at a flow rate of 35 l/h using a 20 wt % aqueous suspension of a solid active principle (AVE1625) containing 1.2 wt % of stabilizer (PVP/SDS:60/40% w/w) (Table II). The AVE1625 is N-[1-[bis(4-chlorophenyl)methyl]azetidin-3-yl]-N-(3,5-difluorophenyl)methanesulfonamide having the CAS No. 358970-97-5,

TABLE II

| | valve made of | | |
|---|---|---|---|
| | | tungsten carbide | |
| | zirconium | coated with titanium | |
| | oxide | nitride | silicon nitride |
| ref. | VRT 769 | VRT 770 | VRT 783 |
| run time [h] | 12.1 | 3.75 | 15.65 |
| observations | breakage of | erosion of valve seat | nothing to report |
| | impact ring | and drop in pressure | |

These tests show that the valve made of silicon nitride is very resistant and does not become damaged during preparation of the nanosuspension.

Following these tests, the silicon nitride valve was kept and used in various tests on the NS2006 without the mechanical properties being impaired, as can be seen from the results of Table III.

TABLE III

| | working | working | |
|---|---|---|---|
| | pressure | flow rate | run time |
| test reference | [bar] | [l/h] | [hours] |
| VRT778 | 1400 | 44.65 | 7.2 |
| VRT779 | 600 | 62.43 | 6.0 |
| VRT780 | 800 | 57.99 | 6.25 |
| VRT781 | 1000 | 52.86 | 6.5 |
| VRT782 | 1200 | 48.60 | 6.5 |
| VRT783 | 1400 | 44.65 | 15.65 |
| VRT784 | 1400 | 44.65 | 6.5 |
| VRT787 | 1400 | 44.48 | 6.55 |
| VRT 789 | 1400 | 42.90 | 5.28 |
| VRT792 | 1400 | 42.73 | 6.5 |
| VRT796 | 1400 | 42.80 | 2.05 |
| VRT797 | 1400 | 41.29 | 5 |
| VRT798 | 1400 | 40.68 | 6.08 |
| VRT799 | 1400 | 40.91 | 6.02 |
| total | | | 92.08 |

For all the tests in Table III, the valve withstood the test and no drop in pressure was noted, a pressure drop being a sign that the magnitude of the gap through which the dispersion must past has increased, and therefore a sign that the valve is impaired. The total number of hours for which the valve was run without any change in valve is therefore at least 92.08 h.

Tests were also conducted on a larger scale (1400 bar, at a flow rate 300 l/h, using NS3024). With the valve made of zirconium oxide the test was stopped on 3 occasions because the valve broke after around 5 hours of running in each instance. The valve made of silicon nitride which had already run for 92.08 h ran for a further 10 h without any particular problem. Moreover, it displayed a better grinding efficiency than the valve made of zirconium oxide.

### CONCLUSIONS

The study demonstrates very good mechanical integrity (no erosion over a long period of time) of the valve made of silicon nitride by comparison with the valves made of zirconium oxide and of tungsten carbide coated with titanium nitride, and did so on two scales (35 and 300 l/h). Moreover, the valve demonstrated greater grinding efficiency by comparison with the valve made of zirconium oxide.

A valve made of silicon nitride, more particularly of the KERSIT® 301 or equivalent material, can therefore be used advantageously in “piston-gap” HPH technology for the preparation of pharmaceutical formulations containing an active principle in a state of nanoparticles dispersed in water and stabilized by at least one stabilizer, the nanoparticles having a mean diameter smaller than 1000 nm and more generally of between 1000 nm and 20 nm.

1. A homogenizing valve comprising a valve piston, an impact ring and a valve seat for the preparation of a nanosuspension of a solid pharmaceutical active principle using piston-gap high-pressure technology, wherein said valve seat, partially, completely or the exterior surface thereof, said valve piston, completely or the exterior surface thereof and optionally said impact ring, completely or the exterior surface thereof, are made of material comprising sintered or hot-pressed silicon nitride.

2. The homogenizing valve of claim 1, wherein said material comprises over 75% (by weight), sintered or hot-pressed silicon nitride.

3. The homogenizing valve of claim 1, wherein said material comprises, by weight, from 80 to 90% sintered or hot-pressed silicon nitride and from 0 to 20% of component(s) chosen from Al₂O₃, Y₂O₃, TiO₂ or Nd₂O₃.

4. The homogenizing claim 3, wherein said material is a hot-pressed silicon nitride consisting, by weight, of 88.5% silicon nitride and of 11.5% Al₂O₃, Y₂O₃, Nd₂O₃, TiO₂ and having the following properties: density>3.25; bending strength>800 MPa; hardness: 1450 Hv; toughness: 7 MPa·m^{1/2}.

5. The homogenizing valve of claim 1, wherein the valve piston is completely made of sintered or hot-pressed silicon nitride.

6. The homogenizing valve of claim 1, wherein the exterior surface of the valve piston is made of sintered or hot-pressed silicon nitride.

7. The homogenizing valve of claim 1, wherein the valve seat is completely made of sintered or hot-pressed silicon nitride.

8. The homogenizing valve of claim 1, wherein the exterior surface of the valve seat is made of sintered or hot-pressed silicon nitride.

9. The homogenizing valve of claim 1, wherein, the internal part of the valve seat is made of sintered or hot-pressed silicon nitride.

10. The homogenizing valve of claim 9, wherein the external part of the valve seat is made of stainless steel.

11. A method of preparing a nanosuspension of a solid pharmaceutical active principle using piston-gap high-pressure homogenization technology, comprising:
pumping a dispersion of the active principle in a liquid phase to which at least one stabilizer/surfactant has been added;
compressing said dispersion to a pressure ranging from 100 to 2000 bar;
expanding said dispersion through a homogenizing valve comprising a valve piston, an impact ring and a valve seat, wherein said valve seat, partially, completely or the exterior surface thereof, said valve piston, completely or the exterior surface thereof and optionally said impact ring, completely or the exterior surface thereof, are made of a material comprising sintered or hot-pressed silicon nitride.

12. The method as claimed in claim 8, wherein a recirculation loop allows the dispersion to be recirculated through the homogenizing valve several times.

13. The homogenizing valve of claim 1, wherein said material comprises over 80% sintered or hot-pressed silicon nitride.

14. The homogenizing valve of claim 1, wherein said material comprises over 85% sintered or hot-pressed silicon nitride.
